(19) United States
(12) Patent Application Publication
(10) Pub. No.: US 2008/0081353 A1
(43) Pub. Date: Apr. 3, 2008

(54) Production of recombinant human collagen

(75) Inventors: Nazrul Islam, Ste-Foy (CA); Francine Goulet, Ste-Foy (CA); Francine Goulet, Ste-Foy (CA); Ioana Diana Napa, Mississauga (CA)

Correspondence Address: GOUDREAU GAGE DUBUC, 2000 MCGILL COLLEGE, SUITE 2200, MONTREAL, QC, H3A 3H3, US

(73) Assignee: UNIVERSITE LAVAL

(21) Appl. No.: 11/529,478

(22) Filed: Sep. 29, 2006

(51) Int. Cl. C12P 21/06; C07H 21/04; C12N 5/06; C12N 15/86

(52) U.S. Cl. 435/69.1; 435/348; 435/320.1; 435/456; 530/356; 536/23.5

(57) ABSTRACT

Methods, reagents (e.g. vectors) and host cells for the recombinant production of collagen are described, relating to the recombinant expression of a subunit of a collagen or procollagen and a collagen post-translational enzyme or subunit thereof.

### CROSS REFERENCE TO RELATED APPLICATIONS

This application claims the benefit of U.S. Provisional Patent Application No. 60/721,921 filed Sep. 30, 2005, which is incorporated herein by reference in its entirety.

### FIELD OF THE INVENTION

The present invention relates to the production of polypeptides using recombinant DNA systems. More specifically, the present invention is concerned with the production of human collagen using such systems.

### BACKGROUND OF THE INVENTION

Collagen is the most abundant component of the extracellular matrix, and is generally formed by the assembly of three polypeptide chains to create a trimeric structure. Nineteen different types of collagen have been described, numbered as types I-XIX. For example, type I collagen, found in several tissues such as bone, tendons and skin, is a heterotrimeric molecule comprising two α-1 (I) chains and one α-2(I) chain. Type II collagen is a homotrimeric molecule comprising three α-1(II) chains. Type III collagen, found in skin and vascular tissues, is a homotrimeric molecule comprising three α-1(III) chains.

Various post-translational modifications during collagen biosynthesis have been described, including, for example, the hydroxylation of proline residues to 4-hydroxyproline by the enzyme prolyl 4-hydroxylase, and cleavage of N- and C-propeptides of procollagen by N- and C-proteinase enzymes, respectively. Other reported collagen post-translational enzymes include lysyl oxidase and lysyl hydroxylase.

Human collagen is desirable for a number of therapeutic applications. Its production by recombinant means is attractive for example to obtain greater amounts where insufficient amounts are available from natural sources, as well as to avoid any adverse immune reactions associated with the use of collagen from non-human sources.

There is therefore a continued need to develop systems for the recombinant production of collagen.

### SUMMARY OF THE INVENTION

The invention relates to the recombinant production of collagen.

More specifically, in accordance with the present invention, there is provided a method for producing a recombinant human collagen polypeptide, said method comprising: (a) culturing a host insect cell, wherein said insect cell has been infected, transfected or transformed with a recombinant baculovirus expression vector comprising: (i) a nucleotide sequence which encodes a collagen subunit, operably linked to a first promoter; and (ii) a nucleotide sequence which encodes a collagen post-translational enzyme or subunit thereof, operably linked to a second promoter different from said first promoter; and (b) recovering said collagen polypeptide from said host insect cell culture.

The invention further provides a method for producing a recombinant human procollagen polypeptide, said method comprising: (a) culturing a host insect cell, wherein said insect cell has been infected, transfected or transformed with a recombinant baculovirus expression vector comprising: (i) a nucleotide sequence which encodes a collagen subunit, operably linked to a first promoter; and (ii) a nucleotide sequence which encodes a collagen post-translational enzyme or subunit thereof, operably linked to a second promoter; and (b) recovering the procollagen polypeptide from the host insect cell culture.

In an embodiment, the above-mentioned infected, transfected or transformed host insect cell comprising the recombinant baculovirus expression vector is obtained by a method comprising: (a) transfecting or transforming a first host insect cell with baculovirus DNA and an expression vector comprising: (i) a nucleotide sequence which encodes a collagen subunit, operably linked to a first promoter; and (ii) a nucleotide sequence which encodes a collagen post-translational enzyme or subunit thereof, operably linked to a second promoter; thereby to permit integration of said expression vector into said baculovirus DNA to obtain a recombinant baculovirus expression vector; (b) isolating a nucleic acid molecule comprising the recombinant baculovirus expression vector from said host cell; and (c) transfecting or transforming a second host insect cell with the nucleic acid molecule obtained in (b) thereby to obtain an infected, transfected or transformed host insect cell comprising said recombinant baculovirus expression vector. In an embodiment, the method further comprises (d) culturing the infected, transfected or transformed host insect cell obtained in (c) under conditions suitable for production of recombinant baculovirus; (e) infecting a third host insect cell with the recombinant baculovirus obtained in (d), thereby to obtain an infected, transfected or transformed host insect cell comprising said recombinant baculovirus expression vector.

The invention further provides a recombinant collagen polypeptide obtained by the above-mentioned method.

The invention further provides a recombinant procollagen polypeptide obtained by the above-mentioned method.

The invention further provides a recombinant baculovirus expression vector comprising: (i) a nucleotide sequence which encodes a collagen subunit, operably linked to a first promoter; and (ii) a nucleotide sequence which encodes a collagen post-translational enzyme or subunit thereof, operably linked to a second promoter.

The invention further provides a host insect cell which has been infected, transfected or transformed with the above-mentioned recombinant baculovirus expression vector.

The invention further provides a method for producing a recombinant human collagen or procollagen polypeptide, the method comprising: (a) culturing a host insect cell, wherein the insect cell has been infected, transfected or transformed with a recombinant baculovirus expression vector comprising: (i) a nucleotide sequence which encodes a collagen subunit, operably linked to a p10 promoter; and (ii) a nucleotide sequence which encodes a collagen post-translational enzyme or subunit thereof, operably linked to a polH promoter; and (b) recovering the collagen or procollagen polypeptide from said host insect cell culture.

In an embodiment, the above-mentioned first promoter is a p10 promoter, e.g., comprising the promoter region set forth in FIG. 5 (SEQ ID NO: 9).

In an embodiment, the above-mentioned second promoter is a polyhedron (polH) promoter, e.g., comprising the promoter region set forth in FIG. 5 (SEQ ID NO: 10).

In an embodiment, the above-mentioned collagen subunit is a first collagen subunit and wherein the recombinant baculovirus expression vector further comprises a nucleotide sequence which encodes a second collagen subunit, operably linked to a first promoter. In a further embodiment, the recombinant baculovirus expression vector further comprises a nucleotide sequence which encodes a third collagen subunit, operably linked to a first promoter.

In an embodiment, the above-mentioned subunit of a collagen post-translational enzyme is a first subunit of a collagen post-translational enzyme, and wherein the recombinant baculovirus expression vector further comprises a nucleotide sequence which encodes a second subunit of a collagen post-translational enzyme, operably linked to a second promoter.

In an embodiment, the above-mentioned collagen is selected from collagen types I, II and Ill.

In an embodiment, the above-mentioned collagen is type II collagen and the collagen subunit is a collagen α1 (II) subunit.

In an embodiment, the above-mentioned collagen is type III collagen and the collagen subunit is a collagen α1 (III) subunit

In an embodiment, the above-mentioned collagen is type I collagen, the first collagen subunit is a collagen α1(I) subunit and the second collagen subunit is a collagen α2(I) subunit.

In an embodiment, the above-mentioned collagen post-translational enzyme is selected from prolyl hydroxylase, lysyl oxidase and lysyl hydroxylase. In a further embodiment, the collagen post-translational enzyme is prolyl 4-hydroxylase

In an embodiment, the above-mentioned collagen post-translational enzyme is prolyl 4-hydroxylase and wherein the first subunit of a collagen post-translational enzyme is an alpha subunit of prolyl 4-hydroxylase and wherein the second subunit of a collagen post-translational enzyme is a beta subunit of prolyl 4-hydroxylase.

In a further aspect, the invention provides a method of increasing or enhancing the purity of a collagen preparation, the method comprising incubating the collagen preparation under basic conditions such that the collagen is rendered insoluble in the basic solution, and recovering the insoluble collagen. In an embodiment, the method comprises dialyzing the collagen preparation against a basic solution.

In a further aspect, the invention provides a method of preparing collagen or processing a procollagen, the method comprising treating a procollagen sample with an elastase.

Other advantages and features of the present invention will become more apparent upon reading of the following non-restrictive description of specific embodiments thereof, given by way of example only with reference to the accompanying drawings.

### BRIEF DESCRIPTION OF THE DRAWINGS

FIG. 1: DNA (SEQ ID NO: 1) and polypeptide (SEQ ID NO: 2) sequences of human α-1(I) collagen (Homo sapiens col1-α1: Accession No. NM_{—}000088; GI: 14719826). SEQ ID NO: 2 corresponds to the coding sequence defined by positions 127-4521 in SEQ ID NO: 1.

FIG. 2: DNA (SEQ ID NO: 3) and polypeptide (SEQ ID NO: 4) sequences of human α-2(I) collagen (Homo sapiens collI-α2: Accession No. NM_{—}000089, GI: 48762933), including the 5086 bp sequence coding for pre-pro-collI-α2 set forth in Accession No. Z74616, GI: 1418929. SEQ ID NO: 4 corresponds to the coding sequence defined by positions 472-4572 in SEQ ID NO: 3.

FIG. 3: DNA (SEQ ID NO: 5) and polypeptide (SEQ ID NO: 6) sequences of human prolyl 4-hydroxylase alpha subunit (α-p4H; Homo sapiens p4Hα: 2722 bp sequence set forth in Accession No. M24486 (clone PA-II), GI: 190785). SEQ ID NO: 6 corresponds to the coding sequence defined by positions 119-1723 in SEQ ID NO: 5.

FIG. 4: DNA (SEQ ID NO: 7) and polypeptide (SEQ ID NO: 8) sequences of human prolyl 4-hydroxylase beta subunit (β-p4H; Homo sapiens p4Hβ: 1956 bp sequence set forth in Accession No. X05130, GI: 35654). SEQ ID NO: 8 corresponds to the coding sequence defined by positions 30-1556 in SEQ ID NO: 7

FIG. 5: Sequences of Autographa californica multicapsid nuclear polyhedrosis virus (AcMNPV) p10 (SEQ ID NO: 9) and polH (SEQ ID NO: 10) promoter regions (derived from Autographa californica nucleopolyhedrovirus complete genome Accession No. NC_{—}001623, GI: 9627742), and also indicated in technical materials for plasmid pBAC4x-1™ (Novagen).

FIG. 6. Photomicrographs taken under transmission electron microscope of Sf9 insect cells transfected with recombinant baculovirus, showing (A): viral inclusions within the cytoplasm of the cells and (B): lysis of the cell membrane by the virus.

FIG. 7: Photomicrographs taken under phase contrast microscope of the polymerized recombinant human type I collagen fibrils showing alignment and formation of a structured network, (40×). Panel A shows neo-formed recombinant human type I collagen fibrils in formation, whereas panel B shows a sample prepared from the same batch and digested with pepsin, where no fibrils were detected.

FIG. 8: Scanning electron microscopy image of polymerized recombinant human type I collagen fibers, assembled naturally.

FIG. 9: Results of SDS-PAGE (under reducing conditions) analysis of recombinant human type I collagen chains, showing the purity of recombinant human type I collagen and the expression of the α1- and α2-chains prepared using the method described herein.

FIG. 10: Recombinant procollagen type I resolved by SDS-PAGE, transferred on a nitrocellulose paper, followed by Concanavalin-A-biotin and streptavidin-Peroxidase blotting, showing the positive staining of its glycosylated amino acid residues.

### DESCRIPTION OF ILLUSTRATIVE EMBODIMENTS

Applicants describe herein a method of producing collagen using a recombinant expression system. The method is preferably for the production of human collagen.

Applicants have found that collagen may be successfully produced in a recombinant expression system via the use of a single expression vector comprising both the nucleic acid(s) encoding a collagen subunit(s) and the nucleic acid(s) encoding a collagen post-translational enzyme or subunit(s) thereof. Prior to applicants' studies described herein, attempts to produce recombinant collagen entailed the use of multiple expression vectors.

In an embodiment, four nucleic acids (which encode a corresponding polypeptide) may be inserted into a single vector, preferably a baculovirus vector. For example, these four nucleic acids may encode a first collagen subunit, a second collagen subunit, a first subunit of a collagen post-translational enzyme, and a second subunit of a collagen post-translational enzyme, respectively.

In a preferred embodiment, the expression system is a baculovirus/insect cell expression system. This system is advantageous because it provides, among other things, high levels of expression of the recombinant protein with the appropriate post-translational modifications and is amenable to scale-up for large-scale production. Various reagents for baculovirus/insect cell expression systems are known in the art and are commercially available. The Baculovirus Expression Vector System (BEVS) is one of the most powerful and versatile eukaryotic expression systems available. This expression system relies on the generation of recombinant baculoviruses in which viral genes, not essential for viral replication in cell culture, are replaced by DNA sequences of interest, (O'Reilly et al., 1992; Kidd and Emery, 1993). The recombinant viral DNA is typically transfected into Spodoptera frugiperda (Sf9) insect cells, clonal derivative of the fall armyworm Spodoptera frugiperda ovarian cell line, IPLB-Sf21-AE, (Sf21), (Vaughn et al, 1977; Nobiron et al, 2003). The recombinant proteins expressed in the baculovirus system are properly folded, disulphide bonded, oligomerized, and localized in the same subcellular compartment as the authentic protein (Kidd and Emery, 1993). Insect cells are also capable of performing several post-translational modifications such as N- and O-glycosylation, phosphorylation, acylation, amidation, carboximethilation, signal peptide cleavage, and proteolytic cleavage (Matsuura et al., 1987; Nokelainen M., 2000). The sites where these modifications occur are often identical to those of the authentic protein in its native cellular environment (Hoss et al., 1990; Kloc et al., 1991; Kuroda et al., 1990). In addition, insect cells possess a low prolyl-4-hydroxilase activity (Veijola et al., 1994). In this system, expression of the above-noted nucleic acids may be driven by the polyhedron (polH) promoter or the p10 promoter, both of which are known for use in baculovirus expression systems. Multiple copies of these promoters may be used in a vector to express multiple nucleic acids of interest.

In a preferred embodiment, the expression of the above-noted nucleic acids is driven by two different promoters, e.g., respective first and second promoters, such as the polH promoter for the expression of the collagen subunit and the p10 promoter for the expression of the collagen post-translational enzyme or subunit thereof.

Accordingly, in a first aspect, the invention provides a method for producing a recombinant collagen or procollagen polypeptide, such as a human collagen or procollagen polypeptide, said method comprising:
(a) culturing a host cell, such as a host insect cell, wherein the host cell has been infected, transfected or transformed with a recombinant expression vector (e.g., a recombinant baculovirus expression vector) comprising:
   (i) a nucleotide sequence which encodes a collagen subunit, operably linked to a first promoter (e.g., a polyhedron (polH) promoter); and
   (ii) a nucleotide sequence which encodes a collagen post-translational enzyme or subunit thereof, operably linked to a second promoter (e.g. a p10 promoter);
(b) recovering said collagen or procollagen polypeptide from said host cell culture.

In embodiments, the host cell is a eukaryotic host cell selected from an insect cell, a fungal (e.g. yeast) cell, a mammalian cell and a plant cell. In a preferred embodiment the host cell is an insect host cell, such as a Spodoptera frugiperda-derived cell (e.g., Sf9 or Sf21).

In embodiments, single or multiple collagen or procollagen subunits can be expressed using the method of the invention. Therefore, in embodiments, the vector further comprises a nucleotide sequence which encodes a second collagen subunit operably linked to a promoter (e.g., a polH or p10 promoter). In a further embodiment, the vector yet further comprises a nucleotide sequence which encodes a third collagen subunit operably linked to a promoter (e.g., a polH or a p10 promoter).

In an embodiment, the vector further comprises a nucleotide sequence which encodes a further subunit of a collagen post-translational enzyme, operably linked to a promoter (e.g., a polH or a p10 promoter).

In embodiments, the collagen is selected from collagen types I-XIX. The appropriate collagen subunits for expression in each case are known in the art. For example, relevant subunits in respect of certain types of collagen are set forth in Table 1.

TABLE 1
Structures of certain types of collagen

| Type | Structure |
|---|---|
| I | heterotrimeric: two α 1(I) chains; one α 2(I) chain |
| II | homotrimeric: three α 1(II) chains |
| III | homotrimeric: three α 1(III) chains |
| IV | most common form: |
| | heterotrimeric: two α 1(IV) chains; one α 2(IV) chain |
| V | multiple forms, e.g.: |
| | heterotrimeric: two α 1(V) chains; one α 2(V) chain |
| | heterotrimeric: one each of α 1(V), α 2(V) and α 3(V) chains |
| | homotrimeric: three α 1(V) chains |
| VI | heterotrimeric: one each of α 1(VI), α 2(VI) and α 3(VI) chains |
| VII | homotrimeric: three α 1(VII) chains |
| VIII | heterotrimeric: two α 1(VIII) chains; one α 2(VIII) chain (other |
| | structures also described) |
| IX | heterotrimeric: one each of α 1(IX), α 2(IX) and α 3(IX) chains |
| X | homotrimeric: three α 1(X) chains |
| XI | heterotrimeric: one each of α 1(XI), α 2(XI) and α 3(XI) chains |
| XII | homotrimeric: three α 1(XII) chains |
| XIV | homotrimeric: three α 1(XIV) chains |

In embodiments, the collagen is selected from types I, II and Ill. In the case of type II collagen, the vector comprises a nucleotide sequence which encodes a collagen α1(II) subunit, operably linked to a promoter (e.g., a polH or a a p10 promoter). In the case of type III collagen, the vector comprises a nucleotide sequence which encodes a collagen α1(III) subunit, operably linked to a promoter (e.g., a polH or a p10 promoter). In the case of type I collagen the vector comprises a nucleotide sequence which encodes a collagen α1(I) subunit, operably linked to a promoter (e.g., a polH or a p10 promoter), and further comprises a a nucleotide sequence which encodes a collagen α2(I) subunit, operably linked to a promoter (e.g., a polH or a p10 promoter).

In further embodiments, multiple copies of a a nucleotide sequence which encodes a collagen subunit may be included in the vector. For example, in the case of type II collagen (which has a homotrimeric structure), two (or more) copies of a nucleotide sequence encoding a collagen α1(II) subunit may be included in the vector.

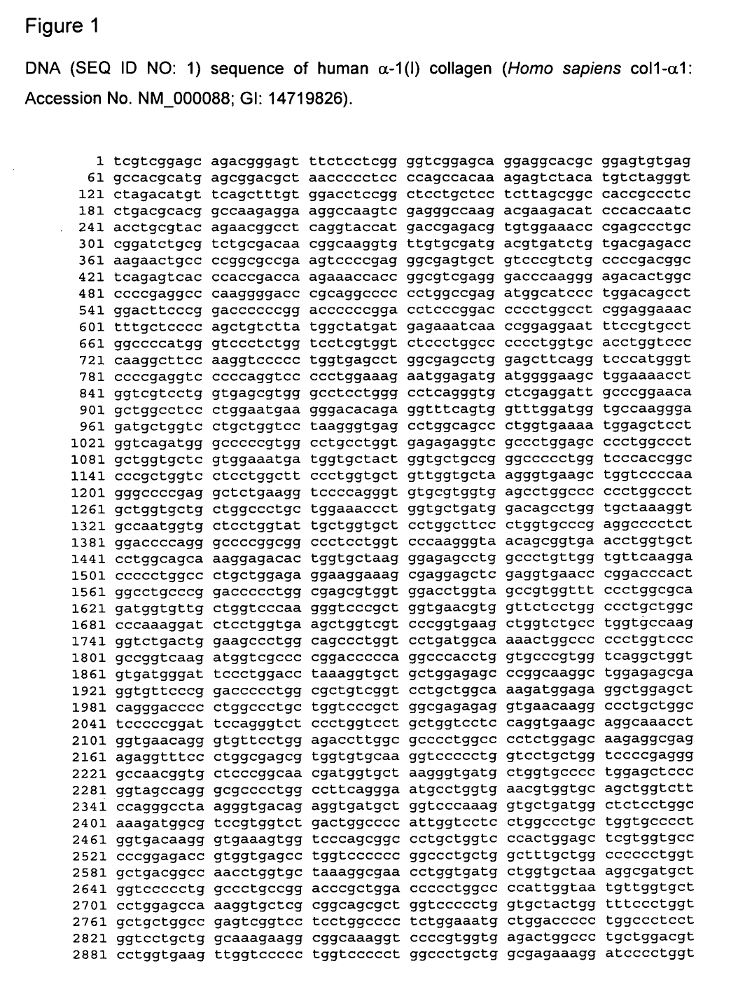

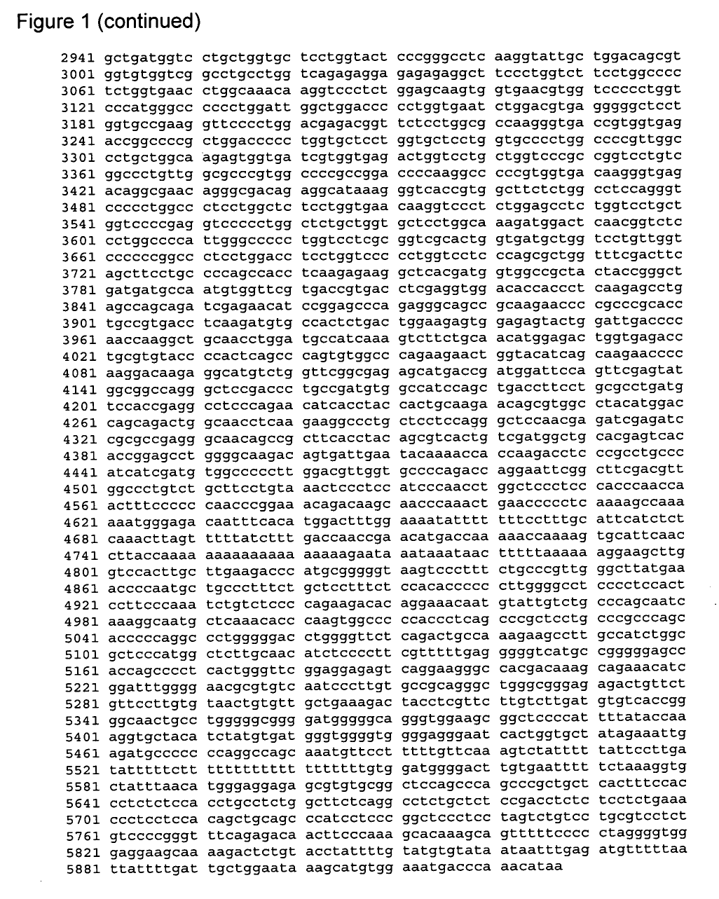

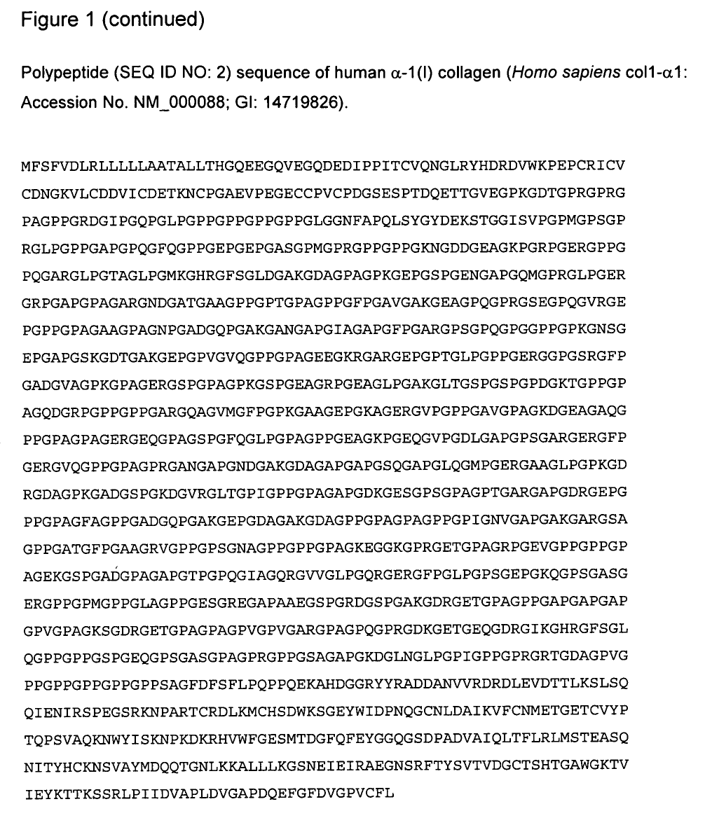

An example of a human collagen α1(I) subunit corresponds to the polypeptide (SEQ ID NO: 2) set forth in FIG. 1, which also sets forth the nucleic acid sequence (SEQ ID NO: 1) encoding the polypeptide. Positions 127-192 of SEQ ID NO: 1 correspond to the signal peptide. Positions 193-4518 of SEQ ID NO: 1 correspond to the proprotein.

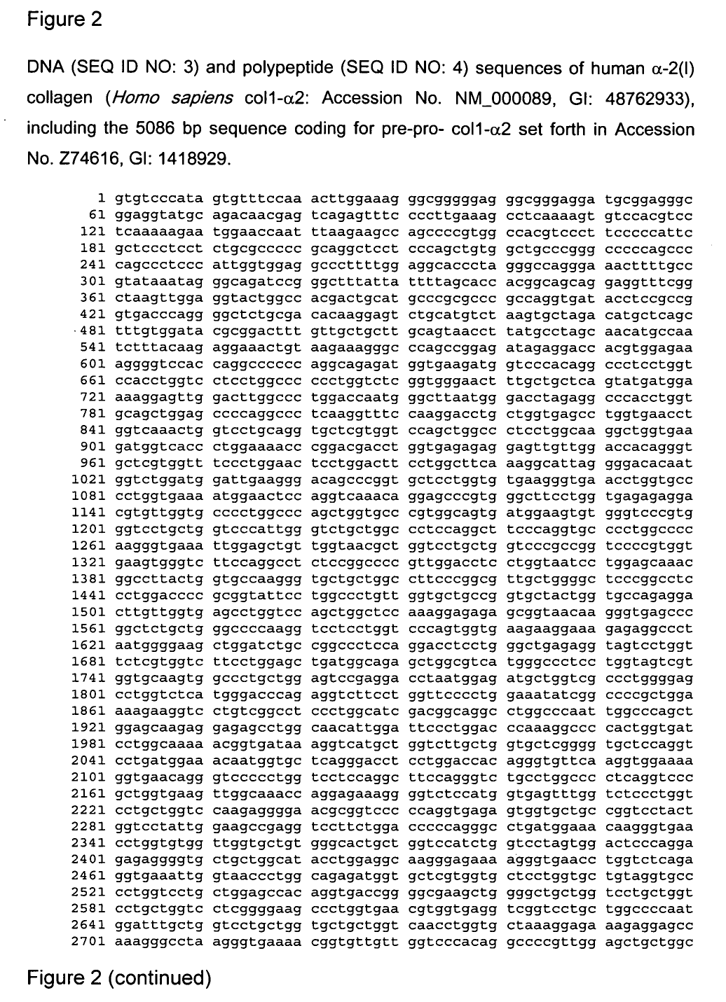

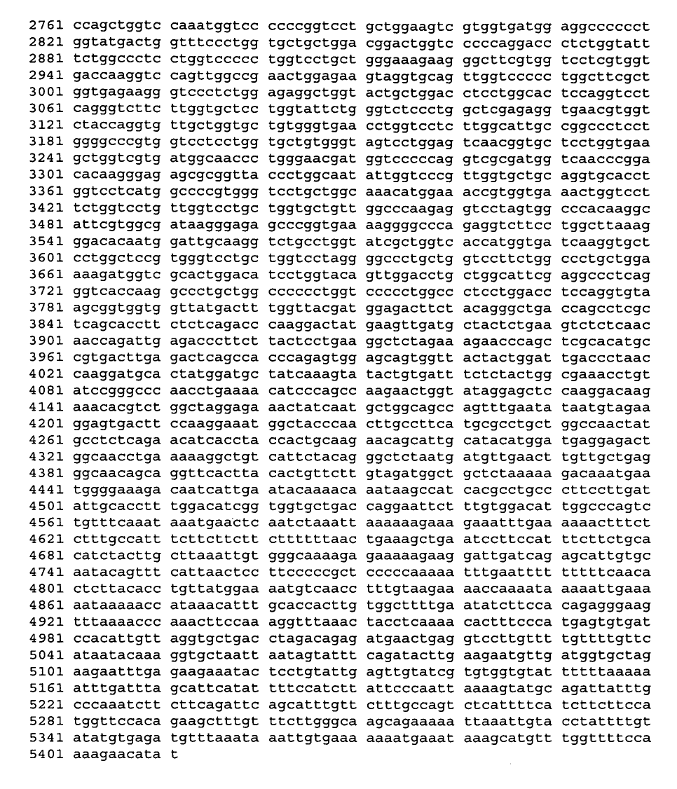

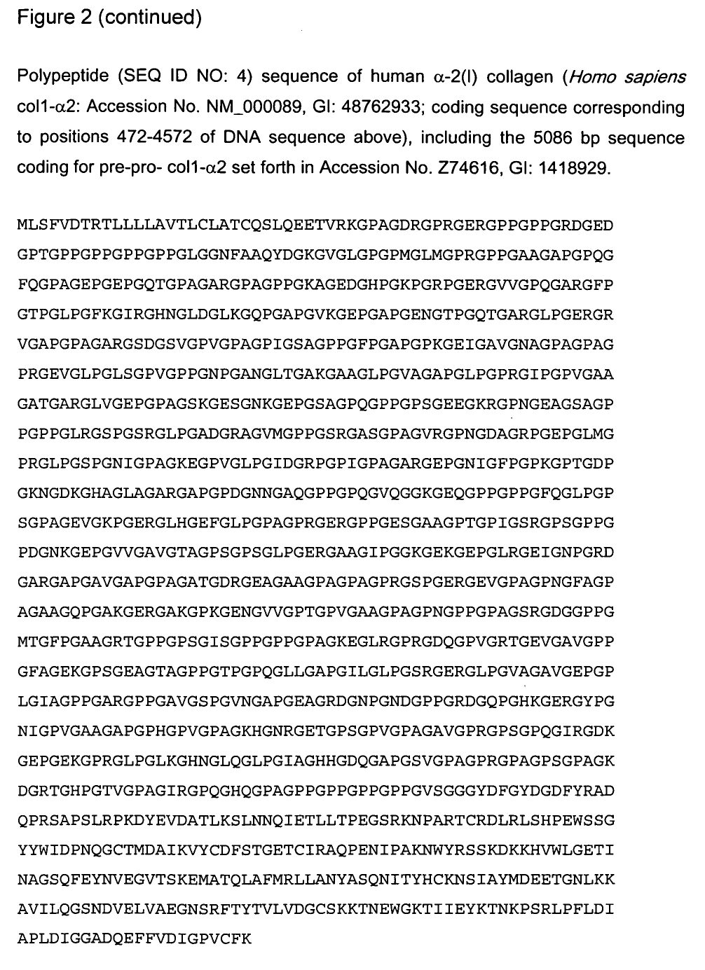

An example of a human collagen α2(I) subunit corresponds to the polypeptide (SEQ ID NO: 4) set forth in FIG. 2, which also sets forth the nucleic acid sequence (SEQ ID NO: 3) encoding the polypeptide.

In embodiments, the collagen post-translational enzyme is selected from prolyl hydroxylase (e.g. prolyl 4-hydroxylase), lysyl oxidase, lysyl hydroxylase, N-proteinase and C-proteinase.

Prolyl 4-hydroxylase is classified under enzyme classification EC 1.14.11.2 and catalyzes the hydroxylation of proline residues to 4-hydroxyproline in the synthesis of collagen or procollagen. The human form comprises two subunits, denoted as alpha and beta subunits. The human alpha-I isoform of the alpha subunit corresponds to for example Genbank accession No. M24486. The human alpha-II isoform of the alpha subunit corresponds to for example Genbank accession No. U90441. The human beta subunit corresponds to for example Genbank accession No. X05130. The vertebrate enzyme is a tetramer comprising two alpha and two beta subunits.

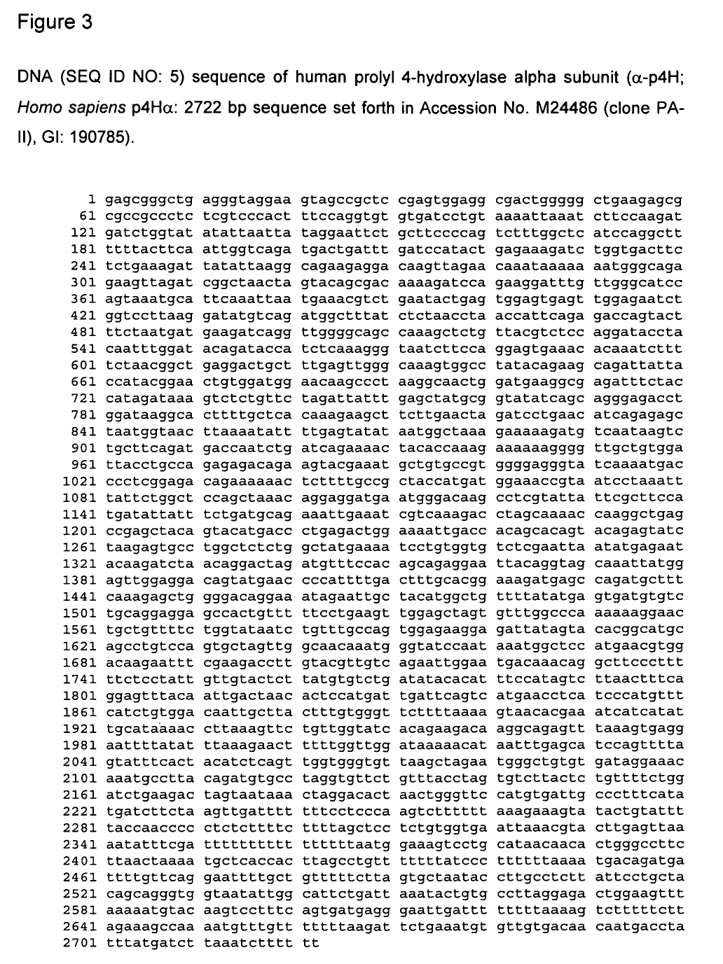

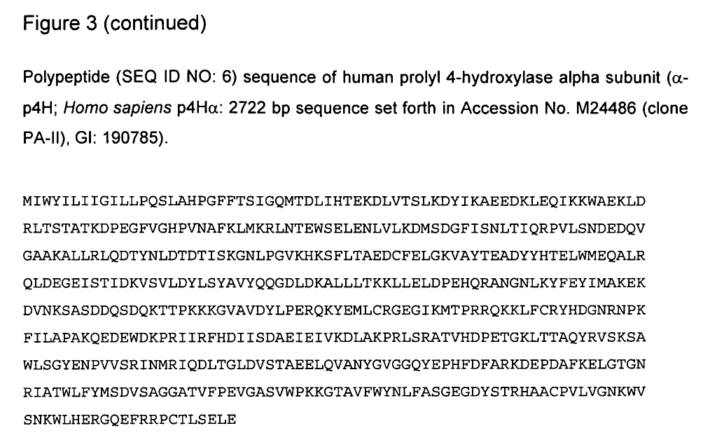

An example of a human prolyl 4-hydroxylase alpha subunit corresponds to the polypeptide (SEQ ID NO: 6) set forth in FIG. 3, which also sets forth the nucleic acid sequence (SEQ ID NO: 5) encoding the polypeptide.

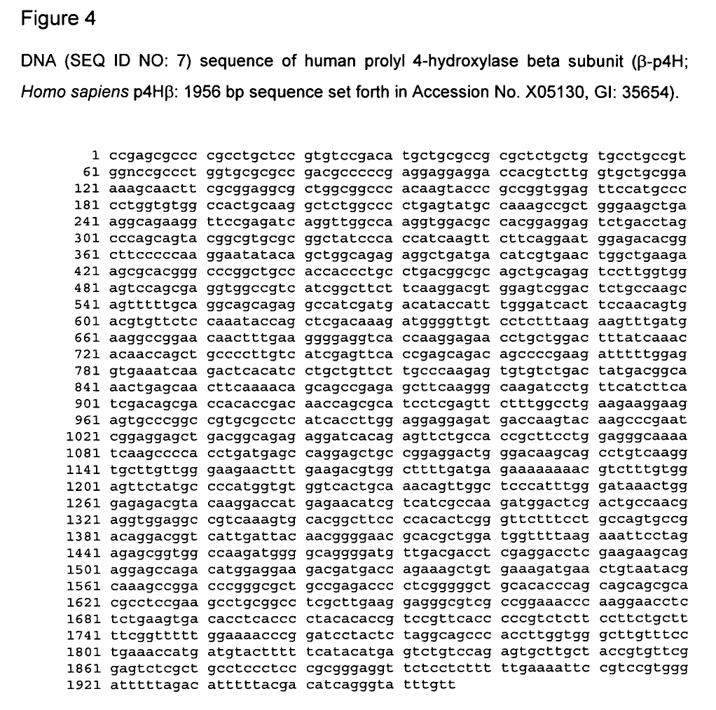

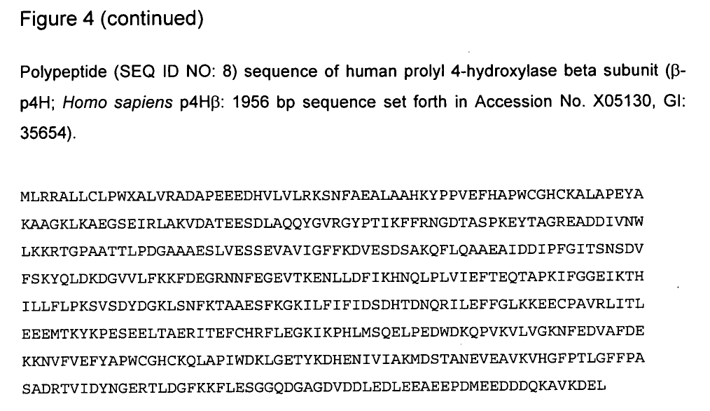

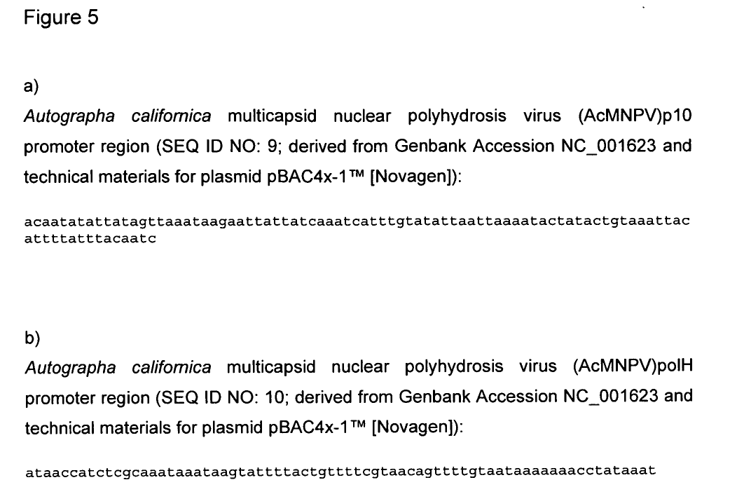

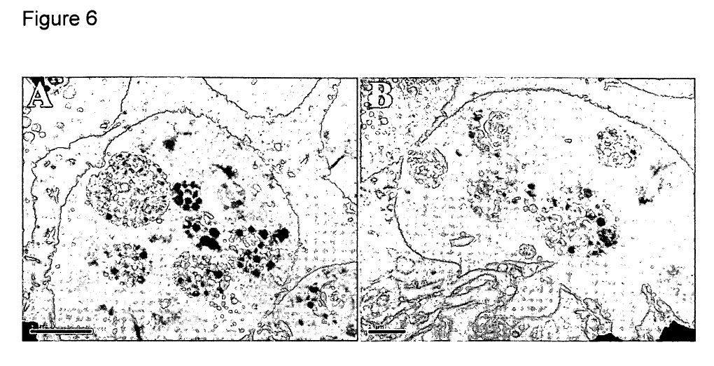

An example of a prolyl 4-hydroxylase beta subunit corresponds to the polypeptide (SEQ ID NO: 8) set forth in FIG. 4, which also sets forth the nucleic acid sequence (SEQ ID NO: 7) encoding the polypeptide. Positions 30-80 of SEQ ID NO: 7 correspond to the signal peptide.

Lysyl hydroxylase is classified under classification EC 1.14.11.4 and catalyzes the hydroxylation of Lys residues in the -X-Lys-Gly- triplet motif of collagens. The enzyme is a homodimer of two alpha subunits. Various forms of the human enzyme correspond to for example Swiss-Prot accession Nos. Q02809, O00469 and O60568.

In the studies described herein, applicants have determined that post-translational processing of collagen may be effected by treatment with elastase. Therefore, in a further aspect, the invention provides a method of preparing collagen or processing a procollagen, said method comprising treating said procollagen sample with an elastase enzyme.

A preferred expression system to be used in the method of the invention is the baculovirus/insect cell expression system, whereby the expression vector comprising the above-noted nucleic acids is introduced into a host insect cell using a baculovirus construct. The host cell is thus cultured under conditions suitable for polypeptide production. An example of such a system utilizes the Autographica californica nuclear polyhydrosys virus (AcNPV), which grows in Spodoptera frugiperda cells. The nucleic acid(s) encoding the recombinant polypeptide(s) of interest (operably linked to an appropriate promoter for expression in the host cell) can be inserted into a non-essential region of AcNPV such as the polyhedron gene. In an embodiment, recombination of these nucleic acids into the non-essential region can result in the replacement or disruption of a marker gene, such as the lacz gene (β-galactosidase), thus allowing selection of recombinants based on the absence of the marker's activity. Such selection is sometimes referred to as a “plaque assay”, as plaques may be selected on the basis of the absence or presence of marker activity. Such recombinant viruses may be used to infect the host insect cell for expression of the polypeptide(s) encoded by the inserted nucleic acid(s).

Preparation of such recombinant viruses typically entails the co-infection of linear viral DNA and a vector comprising the nucleic acid(s) encoding the recombinant polypeptide(s) of interest (operably linked to an appropriate promoter for expression in the host cell) into a host insect cell, whereby recombination results in the insertion of the nucleic acid(s) into the viral DNA. Recombinant virus produced may be identified by plaque assay, which is typically repeated to perform a second round of plaque purification. Ultimately, a stock of recombinant virus is obtained and used to infect host insect cells for polypeptide expression.

In an embodiment, the method comprises an isolation or amplification step, whereby the recombinant viral DNA comprising the nucleic acid(s) encoding the polypeptide(s) of interest (operably linked to an appropriate promoter for expression in the host cell) is isolated or obtained by amplification (e.g. by polymerase chain reaction [PCR]). The isolated or amplified recombinant viral DNA may then be directly introduced (e.g. transfected or transformed) into a host insect cell. Applicants have found that the use of such an additional isolation or amplification step further allows for the efficient preparation of host insect cells for the production of recombinant collagen or procollagen, rather than relying on baculovirus infection alone. Recombinant virus obtained from, for example, the culture medium of such host insect cells may be used as a viral stock to infect other host insect cells for further recombinant polypeptide production.

Accordingly, in an embodiment, the above-mentioned infected, transfected or transformed host insect cell comprising said recombinant baculovirus expression vector is obtained by a method comprising:

(a) transfecting or transforming a first host insect cell with baculovirus DNA and an expression vector comprising: (i) a nucleotide sequence which encodes a collagen subunit, operably linked to a promoter (e.g., a polH promoter); and (ii) a nucleotide sequence which encodes a collagen post-translational enzyme or subunit thereof, operably linked to a promoter (e.g., a p10 promoter); thereby to permit integration of said expression vector into said baculovirus DNA to obtain a recombinant baculovirus DNA expression vector;
(b) isolating a nucleic acid molecule comprising said recombinant baculovirus DNA expression vector from said host cell; and
(c) transfecting or transforming a second host insect cell with said nucleic acid molecule obtained in (b) thereby to obtain an infected, transfected or transformed host insect cell comprising said recombinant baculovirus expression vector.

In a further embodiment, the just-noted method further comprises: (d) culturing said infected, transfected or transformed host insect cell obtained in (c) above under conditions suitable for production of recombinant baculovirus; and (e) infecting a third host insect cell with the recombinant baculovirus obtained in (d) above, thereby to obtain an infected, transfected or transformed host insect cell comprising said recombinant baculovirus expression vector.

The invention further provides a recombinant collagen or procollagen polypeptide obtained by the above-mentioned method.

The invention further provides the above-mentioned recombinant expression vector. In an embodiment, the expression vector is a recombinant baculovirus DNA expression vector.

The invention further provides a host cell, such as an insect host cell, which has been infected, transfected or transformed with the above-mentioned recombinant viral DNA expression vector.

“p10 promoter” as used herein refers to a nucleic acid sequence derived from the Autographa californica multicapsid nuclear polyhedrosis virus (AcMNPV) which can modulate the transcription of the AcMNPV p10 gene. Details of the p10 promoter are set forth in Autographa californica nucleopolyhedrovirus complete genome Accession No. NC_{—}001623, GI: 9627742, as well as in the technical materials for plasmid pBAC4x-1™ (Novagen).

“polH promoter” or “polyhedron promoter” as used herein refers to a nucleic acid sequence derived from the AcMNPV which can modulate the transcription of the AcMNPV polyhedron gene. Details of the polH promoter are set forth in Autographa californica nucleopolyhedrovirus complete genome Accession No. NC_{—}001623, GI: 9627742, as well as in the technical materials for plasmid pBAC4x-1™ (Novagen).

“Collagen” as used herein refers to any of the known collagen types (I-XIX) as well as any variants as described herein, and includes single chain, heterotrimeric and homotrimeric molecules of collagen. “Procollagen” as used herein is similarly defined and refers to any of the known procollagen as well as any variants as described herein, and includes single chain, heterotrimeric and homotrimeric molecules of procollagen, but differs from collagen in that it additionally comprises N-terminal and/or C-terminal peptides which are cleaved off for example by N-proteinase and/or C-proteinase enzymes.

As noted above, an isolated nucleic acid, for example a nucleic acid sequence encoding a polypeptide of the invention (e.g., a collagen or procollagen subunit; a collagen post-translational enzyme or subunit thereof), or homolog, fragment or variant thereof, may further be incorporated into a vector, such as a recombinant expression vector. In an embodiment, the vector will comprise transcriptional regulatory sequences or a promoter operably linked to a nucleic acid comprising a sequence capable of encoding a peptide compound, polypeptide or domain of the invention. A first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequences. Generally, operably linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous. “Transcriptional regulatory sequence/element” is a generic term that refers to DNA sequences, such as initiation and termination signals, enhancers, and promoters, splicing signals, polyadenylation signals which induce or control transcription of protein coding sequences with which they are operably-linked. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain Shine-Dalgarno sequences in addition to the −10 and −35 consensus sequences.

As noted above, the invention relates to the recombinant production of collagen or procollagen. Thus, various nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny or cell or genome with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e., by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

The recombinant polypeptides of the invention may also be expressed in the form of a suitable fusion protein, comprising an amino acid sequence of a polypeptide of the invention linked to further polypeptide sequence (e.g. a heterologous sequence). Such fusion proteins are typically produced by expression of recombinant nucleic acids encoding them. In embodiments, the further polypeptide sequence may confer various functions such as to facilitate cellular localization/secretion, detection and purification (e.g. via affinity methods).

The terminology “amplification pair” refers herein to a pair of oligonucleotides (oligos), which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification. As commonly known in the art, the oligos are designed to bind to a complementary sequence under selected conditions.

Oligonucleotide probes or primers of the present invention may be of any suitable length, depending on the particular assay format and the particular needs and targeted sequences employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybrizidation thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid or polypeptide sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. Similarly, “substantially complementary” nucleic acids are nucleic acids in which the complement of one molecule is “substantially identical” to the other molecule. An “unrelated” or “non-homologous” sequence shares less than 40% identity, though preferably less than about 25% identity, with a nucleic acid or polypeptide of the invention.

Alignment of sequences for comparisons of identity may be conducted using a variety of algorithms and methods, such as those of Smith and Waterman (1981, Adv. Appl. Math 2: 482), Needleman and Wunsch (1970, J. Mol. Biol. 48:443), Pearson and Lipman (1988, Proc. Natl. Acad. Sci. USA 85: 2444), and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. “Nucleic acid hybridization” or “hybridize” generally refer to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5× Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid (Sambrook et al. 1989, supra). Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Q-replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. Patents are incorporated herein by reference). In general, PCR involves a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analysed to assess whether the sequence or sequences to be detected are present. Detection of the amplified sequence may be carried out by visualization following EtBr staining of the DNA following gel electrophoresis, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then translated (translation) into one polypeptide (or protein) or more.

The recombinant expression vector of the present invention can be constructed by standard techniques known to one of ordinary skill in the art and found, for example, in Sambrook et al. (supra). A variety of strategies are available for ligating fragments of DNA, the choice of which depends on the nature of the termini of the DNA fragments and can be readily determined by persons skilled in the art. The vectors of the present invention may also contain other sequence elements to facilitate vector propagation and selection in bacteria and host cells. In addition, the vectors of the present invention may comprise a sequence of nucleotides for one or more restriction endonuclease sites. Coding sequences such as for selectable markers and reporter genes are well known to persons skilled in the art.

A recombinant expression vector comprising a nucleic acid sequence of the present invention may be introduced into a host cell, which may include a living cell capable of expressing the protein coding region from the defined recombinant expression vector. The living cell may include both a cultured cell and a cell within a living organism. Accordingly, the invention also provides host cells containing the recombinant expression vectors of the invention. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into cells via conventional transformation or transfection techniques. The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Suitable methods for transforming or transfecting host cells can for example be found in Sambrook et al. (supra), and other laboratory manuals. “Infection” as used herein refers to the introduction of nucleic acids into a cell using a virus or viral vector, such as a baculovirus.

Polypeptides produced by the recombinant methods described herein can be purified according to standard protocols that take advantage for example of the intrinsic properties thereof, such as size and charge (i.e. SDS gel electrophoresis, gel filtration, dialysis, centrifugation, ion exchange chromatography . . . ). In addition, the recombinant polypeptide can be purified via affinity chromatography using polyclonal or monoclonal antibodies or other affinity-based systems (e.g. using a suitable incorporated “tag” in the form of a fusion protein and its corresponding ligand). Its structure can be further modified using one or more enzymes or bioactive compounds.

Applicants have further demonstrated herein that dialysis under basic conditions allows for the efficient purification of collagen. As described in the Examples below, when dialyzed under basic conditions (e.g., at about pH 8.5 [e.g., in a sodium acetate buffer]), assembled collagen fibrils polymerize while most contaminant proteins are solubilized. The collagen can then be recovered by appropriate means (e.g., centrifugation).

Accordingly, in a further aspect, the invention provides a method of enhancing the purity of a collagen preparation, said method comprising incubating the collagen under basic conditions (e.g., dialyzing the preparation against a basic solution), and recovering the collagen by suitable means (e.g., centrifugation, filtration, etc.). “Basic conditions” as used herein refers to conditions exhibiting an average pH greater than ph 7.0. In an embodiment, the pH is greater than or equal to 7.5, in a further embodiment, greater than or equal to 8.0. In an embodiment, the pH is about 8.5. Various buffer systems (e.g. acetate) are known in the art to prepare solutions exhibiting such basic conditions.

In a further embodiment a product of the invention (e.g., a polypeptide [e.g., a collagen polypeptide]) is substantially pure. A compound is “substantially pure” when it is separated from the components that naturally accompany it. Typically, a compound is substantially pure when it is at least 60%, more generally 75% or over 90%, by weight, of the total material in a sample. Thus, for example, a polypeptide that is chemically synthesised or produced by recombinant technology will generally be substantially free from its naturally associated components. A nucleic acid molecule is substantially pure when it is not immediately contiguous with (i.e., covalently linked to) the coding sequences with which it is normally contiguous in the naturally occurring genome of the organism from which the DNA of the invention is derived. A substantially pure compound can be obtained, for example, by extraction from a natural source; by expression of a recombinant nucleic acid molecule encoding a polypeptide compound; or by chemical synthesis. Purity can be measured using any appropriate method such as column chromatography, gel electrophoresis, HPLC, etc.

A homolog, variant and/or fragment of a polypeptide of the invention which retains activity, and nucleic acids encoding such a homolog, variant and/or fragment, may also be used in the methods of the invention. Homologs include polypeptide sequences, which are substantially identical to the amino acid sequence of a a polypeptide of the invention, sharing significant structural and functional homology with a polypeptide of the invention. Variants include, but are not limited to, polypeptides, which differ from a a polypeptide of the invention by any modifications, and/or amino acid substitutions, deletions or additions. Modifications can occur anywhere including the polypeptide backbone, (i.e. the amino acid sequence), the amino acid side chains and the amino or carboxy termini. Such substitutions, deletions or additions may involve one or more amino acids. Fragments include a fragment or a portion of a polypeptide of the invention, or a fragment or a portion of a homolog or variant of a polypeptide of the invention.

The present invention is illustrated in further details by the following non-limiting examples.

### EXAMPLES

### Example 1

### Materials and Methods

pBAC4x-1™ transfer plasmid and Insect GeneJuice™ transfection reagent were obtained from Novagen (EMD Biosciences/Novagen/VWR CANLAB, Mississaga, Ontario, Canada). Clones containing nucleic acids encoding subunits of collagen and prolyl-4-hydroxylase were obtained from American Type Culture Collection (ATCC) as follows: ATCC # 59480 for P4H beta subunit; ATCC # 138677 for P4H alpha subunit; ATCC # 95501 for coil 1 alpha-2 chain; and ATCC # 95499 for coil 1 alpha-1 chain.

### Example 2

### Preparation of Baculovirus Expression Construct

Preparation of the baculovirus expression construct pBacNI-hcoll I was performed by modification of pBAC4x-1™. The β subunit of P4H was inserted first. It was cloned and the cDNA was amplified by PCR with primers tagged to the EcoRI/SpeI ends. The PCR amplified cDNA was ligated to the sites SmaI and SpeI in linearized pBAC4x-1™ Secondly, the coil α-1(I) subunit was inserted via the insertion of an XbaI restriction enzyme fragment containing DNA encoding the coil α-1(I) subunit into the XbaI site of linearized pBAC4x-1™. Thirdly, The coil α-2(I) subunit cDNA was inserted via the insertion of an SphI restriction enzyme fragment containing DNA encoding the coil α-2(I) subunit into the SphI site of the linearized pBAC4x-1™. Subsequently, the α and β subunits of P4H were inserted. The detailed sequence of the final construct, is described in Table 2.

TABLE 2
Description of pBacNI-hcoll I sequence (18,169 bps)

| Sequence | Description |
|---|---|
| 1–1242 | plasmid pBac4x derived sequence |
| 1243–1248 | BgIII site |
| 1249–3004 | P4Hβ sequence (oriented counterclockwise) |
| 3148–3152 | BgIII site |
| 3153–3401 | plasmid pBac4x derived sequence |
| 3402–3406 | Xbal site |
| 3408–7207 | Col1α1(I) sequence and non coding |
| | sequence (oriented counterclockwise) |
| 7208–7213 | Xbal site |
| 7214–7453 | plasmid pBac4x derived sequence |
| 7454–7459 | Smal site |
| 7480–9578 | P4Hα sequence |
| 9579–9584 | Spel site |
| 9585–9774 | plasmid pBac4x derived sequence |
| 9775–9780 | HindIII site |
| 14255–14260 | Sphl site |
| 14261–18169 | plasmid pBac4x derived sequence |

### Example 3

### Expression, Maturation and Purification of Recombinant Human Collagen

Infected Sf9 cells were grown for 2-6 days in Grace's medium supplemented with ascorbic acid (50 ug/ml) to stimulate collagen synthesis. sf9 cells (suspension of 1 L) were centrifuged to obtain a pellet of cells that contain human recombinant procollagen. The cell pellet was resuspended in about 100 ml of 50 mM Tris-HCl buffer containing 0.2 M NaCl, at pH 7.4. The cells were broken mechanically to liberate pro-collagen (e.g. freezing-thawing twice at −20° C. and 4° C., respectively). Since the extract also contains DNA, coming out of the broken cells, that can provoke DNA-pro-collagen aggregates, DNAse treatment was used to eliminate the DNA.

The procoll/coll suspension was then digested with elastase at 4° C., for 2-3 hrs by adding half volume of 50 mM Tris-HCL, pH 8.5 containing elastase (1-2 mg/ml). After this incubation period, the assembled collagen fibrils were dialyzed against a sodium acetate buffer pH 8.5 for 72 hrs at 4° C. The white fibrils polymerize, while most contaminant proteins were solubilized during dialysis.

After the completion of the dialysis, the collagen was centrifuged for 20 min at 6000 rpm and the pellet was rinsed twice with megapure water, centrifuging each time to recuperate the pellet. A protease inhibitor cocktail was added to the fibrils. The collagen was solubilized in citric acid 0.075M, pH 3.7, overnight, and the residual contaminant proteins that are precipitated were discarded by centrifugation (pellet). The supernatant, containing the acid-solubilized collagen, was dialysed against phosphate buffer 0.02M, pH 9.2 to 9.5, at 4° C. The fibrils slowly precipitated within 2-3 days. The fibrils were centrifuged, washed 3 times and resuspended in megapure water. The suspension was frozen at −86° C. and lyophilized.

In some experiments, the procoll/coll suspension was then precipitated with ammonium sulfate for about 2 hrs at 4° C. and centrifuged to obtain a pellet of proteins. The pellet was resuspended in Tris-HCl buffer containing 0.2 M NaCl, at pH 7.4 and the suspension was dialyzed against a acetic acid (1:1000 or 0.5M) for 72 hrs at 4° C. Then, the suspension was frozen at −80° C. and lyophilized.

### Example 4

### Characterization of Recombinant Human Collagen

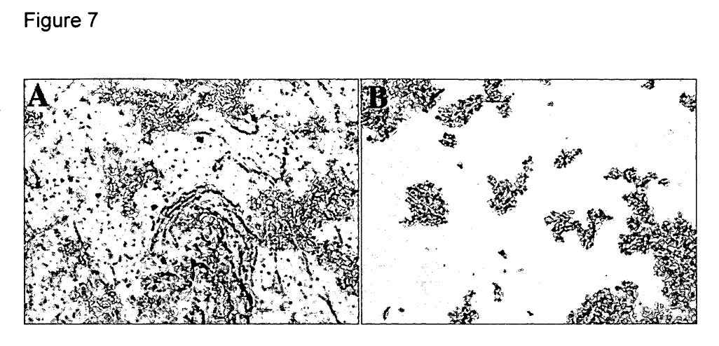

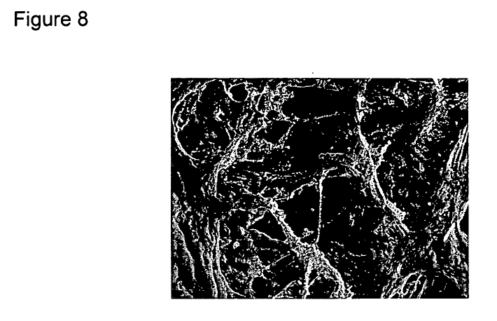

The total amino acid composition, including the percentage of the collagen content in proline, hydroxy-proline, lysine and hydroxy-lysine, and a partial amino acid sequencing of the final product may be performed. The material for analysis may be cut enzymatically before being analyzed. Electron microscopy analyses can reveal the length, the periodicity and the overall organization of the collagen fibers, and thermostability can be evaluated also (Fertala et al., 1994). For example, FIGS. 7 and 8 show results of microscopic analysis of collagen produced according to the method described herein.

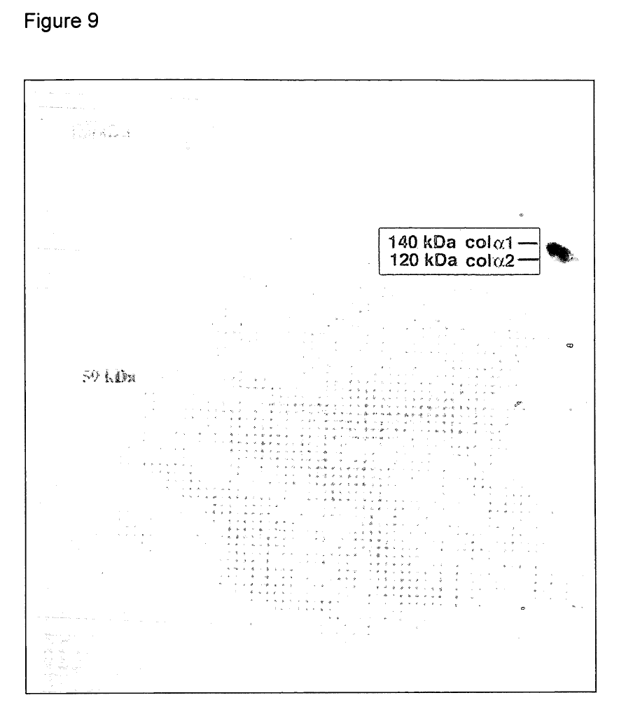

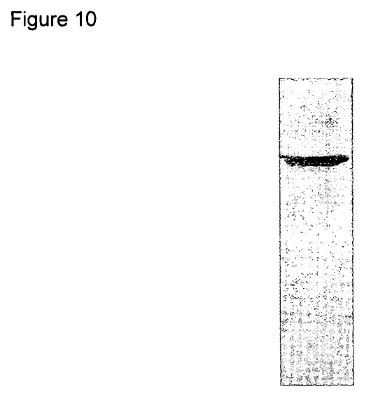

The gycosylation of procollagen can be assessed by testing its affinity with lectins, such as Concanavalin A, that specifically binds glusose and mannose residues. The purity, the respective molecular weights and amounts of α-1 and α-2 chains of the processed collagen can be analyzed on SDS-PAGE. The confirmation of the nature of the collagen can be tested on Western blots, using antibodies directed specifically against human type I collagen. For example, FIG. 9 shows results of SDS-PAGE analysis of collagen produced according to the method described herein. FIG. 10 shows SDS-PAGE analysis together with Concanavalin A-based staining of collagen produced according to the method described herein.

The capacity of the collagen to polymerise into a gel can be assessed by solubilizing the collagen in acetic acid 1:1000 and bring the solution to physiological pH (7.2-7.5).

The present description refers to a number of documents, the content of which is herein incorporated by reference in their entirety.

Although the present invention has been described hereinabove by way of specific embodiments thereof, it can be modified, without departing from the spirit and nature of the subject invention as defined in the appended claims.

### REFERENCES

Behera A K, Kumar M, Bansal A, Bansal O B, Das R H. Expression of lacZ reporter gene under the control of the polyhedrin promoter of Spodoptera litura nuclear polyhedrosis virus. Gene 190: 145-150, 1997.
Bulleid N J, John D C A, Kadler K E. Recombinant expression systems for the production of collagen. Biochem. Soc. Transact. 28:350-353, 2000.
Fertala A, Sieron A L, Hojima Y, Ganguly A, Prockop D J. Self-assembly into fibrils of collagen I1 by enzymic cleavage of recombinant procollagen II. J. Biol. Chem. 269: 11584-11589, 1994.
Hoss A, Moarefi I, Scheidtmann K H, Cisek L J, Corden J L, Dornreiter I, Arthur A K, Fanning E. Altered phosphorylation pattern of simian virus 40 T antigen expressed in insect cells by using a baculovirus vector. J. Virol. 64: 4799-4807, 1990.
Hojima Y, Behta B, Romanic A M, Prockop D J Cadmium ions inhibit procollagen C-proteinase and cupric ions inhibit procollagen N-proteinase. Matrix Biol. 14:113-120, 1994a.
Hojima Y, Morgelin M M, Engel J, Boutillon M M, van der Rest M, McKenzie J, Chen G C, Rafi N, Romanic A M, Prockop D J. Characterization of type I procollagen N-proteinase from fetal bovine tendon and skin. Purification of the 500-kilodalton form of the enzyme from bovine tendon. J Biol. Chem. 269:11381-11390, 1994b.
Kidd I M, Emery V C. The use of baculoviruses as expression vectors. Appl. Biochem. Biotechnol. 42:137-159, 1993.
Kloc M, Reddy B, Crawford S, Etkin L D. A novel 110-kDa maternal CAAX box-containing protein from Xenopus is palmitoylated and isoprenylated when expressed in baculovirus. J. Biol. Chem. 266: 8206-8212, 1991.
Kuroda K, Geyer H, Geyer R, Doerfler W, Klenk H D. The oligosaccharides of influenza virus hemagglutinin expressed in insect cells by a baculovirus vector. Virology 174: 418-429, 1990.
Lamberg A, Helaakoski T, Myllyharju J, Peltonen S, Notbohm H, Pihlajaniemi T, Kivirikko K I. Characterization of human type III collagen expressed in a baculovirus system. Production of a protein with a stable triple helix requires coexpression with the two types of recombinant prolyl 4-hydroxylase subunit. J. Biol. Chem. 271: 11988-11995, 1996.
Matsuura Y, Possee R D, Overton H A, Bishop D H. Baculovirus expression vectors: the requirements for high level expression of proteins, including glycoproteins. J Gen Virol. 68: 1233-1250, 1987.
Moschcovich L, Bernocco S, Font B, Rivkin H, Eichenberger D, Chejanovsky N, Hulmes D J, Kessler E. Folding and activity of recombinant human procollagen C-proteinase enhancer. Eur. J. Biochem. 268: 2991-2996, 2001.
Nobiron I, O'Reilly D R, Olszewski J A. Autographa californica nucleopolyhedrovirus infection of Spodoptera frugiperda cells: a global analysis of host gene regulation during infection, using a differential display approach. J. Gen. Virol. 84:3029-3039, 2003.
Nokelainen M. Recombinant human collagens: characterization of type ii collagen expressed in insect cells and production of types I-III collagen in the yeast pichia pastoris. Department of medical biochemistry. Faculty of medicine, University of Oulu (Findland), Oulu University press, pp. 70, 2000.
O'Reilly D, Miller L K, Luckow V A. Baculovirus expression vectors: A Laboratory manual. W.H. Freeman and Co., New York, 1992.
Possee R D, Howard S C. Analysis of the polyhedrin gene promoter of the Autographa californica nuclear polyhedrosis virus. Nucleic Acids Res. 15: 10233-1048, 1987.
Romanic A M, Adachi E, Kadler K E, Hojima Y, Prockop D J. Copolymerization of pNcollagen III and collagen I. pNcollagen III decreases the rate of incorporation of collagen I into fibrils, the amount of collagen I incorporated, and the diameter of the fibrils formed. J. Biol. Chem. 266:12703-12709, 1991.
Romanic A M, Adachi E, Hojima Y, Engel J, Prockop D J. Polymerization of pNcollagen I and copolymerization of pNcollagen I with collagen I. A kinetic, thermodynamic, and morphologic study. J Biol. Chem. 267: 22265-71, 1992.
Vaughn J L, Goodwin R H, Tompkins G J, McCawley P. The establishment of two cell lines from the insect Spodoptera frugiperda (Lepidoptera; Noctuidae). In Vitro 13:13-17, 1977.
Veijola J, Koivunen P, Annunen P, Pihlajaniemi T, Kivirikko K I. Cloning, baculovirus expression, and characterization of the alpha subunit of prolyl 4-hydroxylase from the nematode Caenorhabditis elegans. This alpha subunit forms an active alpha beta dimer with the human protein disulfide isomerase/beta subunit. J. Biol. Chem. 269: 26746-26753, 1994.
Vlak J M, Klinkenberg F A, Zaal K J, Usmany M, Klinge-Roode E C, Geervliet J B, Roosien J, van Lent J W. Functional studies on the p10 gene of Autographa californica nuclear polyhedrosis virus using a recombinant expressing a p10-beta-galactosidase fusion gene. J. Gen. Virol. 69:765-776, 1988.
Weyer U, Possee R D. Functional analysis of the p10 gene 5′ leader sequence of the Autographa californica nuclear polyhedrosis virus. Nucleic Acids Res. 16: 3635-3653, 1988.
Weyer U, Knight S, Possee R D. Analysis of very late gene expression by Autographa californica nuclear polyhedrosis virus and the further development of multiple expression vectors. J Gen Virol. 71: 1525-1534, 1990.
Weyer U, Possee R D. A baculovirus dual expression vector derived from the Autographa californica nuclear polyhedrosis virus polyhedrin and p10 promoters: co-expression of two influenza virus genes in insect cells. J. Gen. Virol. 72: 2967-2974, 1991.

What is claimed is:

1. A method for producing a recombinant human collagen polypeptide, said method comprising:
(a) culturing a host insect cell, wherein said insect cell has been infected, transfected or transformed with a recombinant baculovirus expression vector comprising:
(i) a nucleotide sequence which encodes a collagen subunit, operably linked to a first promoter; and
(ii) a nucleotide sequence which encodes a collagen post-translational enzyme or subunit thereof, operably linked to a second promoter different from said first promoter; and
(b) recovering said collagen polypeptide from said host insect cell culture.

2. The method of claim 1, wherein said first promoter is a p10 promoter.

3. The method of claim 1, wherein said second promoter is a polyhedron (polH) promoter.

4. The method of claim 1, wherein said collagen subunit is a first collagen subunit and wherein said recombinant baculovirus expression vector further comprises a nucleotide sequence which encodes a second collagen subunit, operably linked to a first promoter.

5. The method of claim 4, wherein said recombinant baculovirus expression vector further comprises a nucleotide sequence which encodes a third collagen subunit, operably linked to a first promoter.

6. The method of claim 1, wherein said subunit of a collagen post-translational enzyme is a first subunit of a collagen post-translational enzyme, and wherein said recombinant baculovirus expression vector further comprises a nucleotide sequence which encodes a second subunit of a collagen post-translational enzyme, operably linked to a second promoter.

7. The method of claim 1, wherein said collagen is selected from collagen types I, II and III.

8. The method of claim 4, wherein said collagen is selected from collagen types I, II and III.

9. The method of claim 7 wherein said collagen is type II collagen and said collagen subunit is a collagen α1(II) subunit.

10. The method of claim 7 wherein said collagen is type III collagen and said collagen subunit is a collagen α1(III) subunit

11. The method of claim 8 wherein said collagen is type I collagen, said first collagen subunit is a collagen α1(I) subunit and said second collagen subunit is a collagen α2(I) subunit.

12. The method of claim 1, wherein said collagen post-translational enzyme is selected from prolyl hydroxylase, lysyl oxidase and lysyl hydroxylase.

13. The method of claim 12, wherein said collagen post-translational enzyme is prolyl 4-hydroxylase

14. The method of claim 6, wherein said collagen post-translational enzyme is prolyl 4-hydroxylase and wherein said first subunit of a collagen post-translational enzyme is an alpha subunit of prolyl 4-hydroxylase and wherein said second subunit of a collagen post-translational enzyme is a beta subunit of prolyl 4-hydroxylase.

15. A method for producing a recombinant human procollagen polypeptide, said method comprising:
(a) culturing a host insect cell, wherein said insect cell has been infected, transfected or transformed with a recombinant baculovirus expression vector comprising:
(i) a nucleotide sequence which encodes a collagen subunit, operably linked to a first promoter; and
(ii) a nucleotide sequence which encodes a collagen post-translational enzyme or subunit thereof, operably linked to a second promoter; and
(b) recovering said procollagen polypeptide from said host insect cell culture.

16. The method of claim 15, wherein said first promoter is a p10 promoter.

17. The method of claim 15, wherein said second promoter is a polyhedron (polH) promoter.

18. The method of claim 15, wherein said collagen subunit is a first collagen subunit and wherein said recombinant baculovirus expression vector further comprises a nucleotide sequence which encodes a second collagen subunit, operably linked to a first promoter.

19. The method of claim 18, wherein said recombinant baculovirus expression vector further comprises a nucleotide sequence which encodes a third collagen subunit, operably linked to a first promoter.

20. The method of claim 15, wherein said subunit of a collagen post-translational enzyme is a first subunit of a collagen post-translational enzyme, and wherein said recombinant baculovirus expression vector further comprises a nucleotide sequence which encodes a second subunit of a collagen post-translational enzyme, operably linked to a polH promoter.

21. The method of claim 15, wherein said collagen is selected from collagen types I, II and III.

22. The method of claim 18, wherein said collagen is selected from collagen types I, II and III.

23. The method of claim 21 wherein said collagen is type II collagen and said collagen subunit is a collagen α1(II) subunit.

24. The method of claim 21 wherein said collagen is type III collagen and said collagen subunit is a collagen α1(III) subunit.

25. The method of claim 22 wherein said collagen is type I collagen, said first collagen subunit is a collagen α1(I) subunit and said second collagen subunit is a collagen α2(I) subunit.

26. The method of claim 15, wherein said collagen post-translational enzyme is selected from prolyl hydroxylase, lysyl oxidase and lysyl hydroxylase.

27. The method of claim 26, wherein said collagen post-translational enzyme is prolyl 4-hydroxylase

28. The method of claim 20, wherein said collagen post-translational enzyme is prolyl 4-hydroxylase and wherein said first subunit of a collagen post-translational enzyme is an alpha subunit of prolyl 4-hydroxylase and wherein said second subunit of a collagen post-translational enzyme is a beta subunit of prolyl 4-hydroxylase.

29. The method of claim 1, wherein said infected, transfected or transformed host insect cell comprising said recombinant baculovirus expression vector is obtained by a method comprising:
(a) transfecting or transforming a first host insect cell with baculovirus DNA and an expression vector comprising:
(i) a nucleotide sequence which encodes a collagen subunit, operably linked to a first promoter; and
(ii) a nucleotide sequence which encodes a collagen post-translational enzyme or subunit thereof, operably linked to a second promoter;
thereby to permit integration of said expression vector into said baculovirus DNA to obtain a recombinant baculovirus expression vector;
(b) isolating a nucleic acid molecule comprising said recombinant baculovirus expression vector from said host cell; and
(c) transfecting or transforming a second host insect cell with said nucleic acid molecule obtained in (b) thereby to obtain an infected, transfected or transformed host insect cell comprising said recombinant baculovirus expression vector.

30. The method of claim 29, further comprising:
(d) culturing said infected, transfected or transformed host insect cell obtained in (c) under conditions suitable for production of recombinant baculovirus; and
(e) infecting a third host insect cell with the recombinant baculovirus obtained in (d), thereby to obtain an infected, transfected or transformed host insect cell comprising said recombinant baculovirus expression vector.

31. The method of claim 29, wherein said first promoter is a p10 promoter.

32. The method of claim 29, wherein said second promoter is a polyhedron (polH) promoter.

33. A recombinant collagen polypeptide obtained by the method of claim 1.

34. A recombinant procollagen polypeptide obtained by the method of claim 15.

35. A recombinant baculovirus expression vector comprising:
(i) a nucleotide sequence which encodes a collagen subunit, operably linked to a first promoter; and
(ii) a nucleotide sequence which encodes a collagen post-translational enzyme or subunit thereof, operably linked to a second promoter.

36. The vector of claim 31, wherein said first promoter is a p10 promoter.

37. The vector of claim 31, wherein said second promoter is a polyhedron (polH) promoter.

38. A host insect cell which has been infected, transfected or transformed with the recombinant baculovirus expression vector of claim 31.

39. A method for producing a recombinant human collagen polypeptide, said method comprising:
(a) culturing a host insect cell, wherein said insect cell has been infected, transfected or transformed with a recombinant baculovirus expression vector comprising:
(i) a nucleotide sequence which encodes a collagen subunit, operably linked to a p10 promoter;
(ii) a nucleotide sequence which encodes a collagen post-translational enzyme or subunit thereof, operably linked to a polH promoter; and
(b) recovering said collagen polypeptide from said host insect cell culture.

40. A method for producing a recombinant human procollagen polypeptide, said method comprising:
(a) culturing a host insect cell, wherein said insect cell has been infected, transfected or transformed with a recombinant baculovirus expression vector comprising:
(i) a nucleotide sequence which encodes a collagen subunit, operably linked to a p10 promoter;
(ii) a nucleotide sequence which encodes a collagen post-translational enzyme or subunit thereof, operably linked to a polH promoter; and
(b) recovering said procollagen polypeptide from said host insect cell culture.

41. A method of enhancing the purity of a collagen preparation, said method comprising incubating the collagen preparation under basic conditions such that the collagen is rendered insoluble in the basic solution, and recovering the insoluble collagen.

42. The method of claim 37, wherein said method comprises dialyzing the collagen preparation against a basic solution.

43. A method of preparing collagen or processing a procollagen, said method comprising treating a procollagen sample with an elastase.


SEQUENCE LISTING (10 sequences)

SEQ ID NO: 1 - DNA, 5927 bp, Homo sapiens
  CDS (127)..(4521)
tcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 2 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 3 - DNA, 5411 bp, Homo sapiens
  CDS (472)..(4572)
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 4 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 5 - DNA, 2722 bp, Homo sapiens
  CDS (119)..(1723)
gagcgggctgagggtaggaagtagccgctccgagtggaggcgactgggggctgaagagcgcgccgccctctcgtcccactttccaggtgtgtgatcctgtaaaattaaatcttccaagatgatctggtatatattaattataggaattctgcttccccagtctttggctcatccaggcttttttacttcaattggtcagatgactgatttgatccatactgagaaagatctggtgacttctctgaaagattatattaaggcagaagaggacaagttagaacaaataaaaaaatgggcagagaagttagatcggctaactagtacagcgacaaaagatccagaaggatttgttgggcatccagtaaatgcattcaaattaatgaaacgtctgaatactgagtggagtgagttggagaatctggtccttaaggatatgtcagatggctttatctctaacctaaccattcagagaccagtactttctaatgatgaagatcaggttggggcagccaaagctctgttacgtctccaggatacctacaatttggatacagataccatctcaaagggtaatcttccaggagtgaaacacaaatcttttctaacggctgaggactgctttgagttgggcaaagtggcctatacagaagcagattattaccatacggaactgtggatggaacaagccctaaggcaactggatgaaggcgagatttctaccatagataaagtctctgttctagattatttgagctatgcggtatatcagcagggagacctggataaggcacttttgctcacaaagaagcttcttgaactagatcctgaacatcagagagctaatggtaacttaaaatattttgagtatataatggctaaagaaaaagatgtcaataagtctgcttcagatgaccaatctgatcagaaaactacaccaaagaaaaaaggggttgctgtggattacctgccagagagacagaagtacgaaatgctgtgccgtggggagggtatcaaaatgacccctcggagacagaaaaaactcttttgccgctaccatgatggaaaccgtaatcctaaatttattctggctccagctaaacaggaggatgaatgggacaagcctcgtattattcgcttccatgatattatttctgatgcagaaattgaaatcgtcaaagacctagcaaaaccaaggctgagccgagctacagtacatgaccctgagactggaaaattgaccacagcacagtacagagtatctaagagtgcctggctctctggctatgaaaatcctgtggtgtctcgaattaatatgagaatacaagatctaacaggactagatgtttccacagcagaggaattacaggtagcaaattatggagttggaggacagtatgaaccccattttgactttgcacggaaagatgagccagatgctttcaaagagctggggacaggaaatagaattgctacatggctgttttatatgagtgatgtgtctgcaggaggagccactgtttttcctgaagttggagctagtgtttggcccaaaaaaggaactgctgttttctggtataatctgtttgccagtggagaaggagattatagtacacggcatgcagcctgtccagtgctagttggcaacaaatgggtatccaataaatggctccatgaacgtggacaagaatttcgaagaccttgtacgttgtcagaattggaatgacaaacaggcttccctttttctcctattgttgtactcttatgtgtctgatatacacatttccatagtcttaactttcaggagtttacaattgactaacactccatgattgattcagtcatgaacctcatcccatgtttcatctgtggacaattgcttactttgtgggttcttttaaaagtaacacgaaatcatcatattgcataaaaccttaaagttctgttggtatcacagaagacaaggcagagtttaaagtgaggaattttatatttaaagaactttttggttggataaaaacataatttgagcatccagttttagtatttcactacatctcagttggtgggtgttaagctagaatgggctgtgtgataggaaacaaatgccttacagatgtgcctaggtgttctgtttacctagtgtcttactctgttttctggatctgaagactagtaataaactaggacactaactgggttccatgtgattgccctttcatatgatcttctaagttgatttttttcctcccaagtcttttttaaagaaagtatactgtattttaccaaccccctctcttttcttttagctcctctgtggtgaattaaacgtacttgagttaaaatatttcgattttttttttttttttaatggaaagtcctgcataacaacactgggccttcttaactaaaatgctcaccacttagcctgtttttttatcccttttttaaaatgacagatgattttgttcaggaattttgctgtttttcttagtgctaataccttgcctcttattcctgctacagcagggtggtaatattggcattctgattaaatactgtgccttaggagactggaagtttaaaaatgtacaagtcctttcagtgatgagggaattgattttttttaaaagtctttttcttagaaagccaaaatgtttgtttttttaagattctgaaatgtgttgtgacaacaatgacctatttatgatcttaaatctttttt

SEQ ID NO: 6 - protein, 534 aa, Homo sapiens
MIWYILIIGILLPQSLAHPGFFTSIGQMTDLIHTEKDLVTSLKDYIKAEEDKLEQIKKWAEKLDRLTSTATKDPEGFVGHPVNAFKLMKRLNTEWSELENLVLKDMSDGFISNLTIQRPVLSNDEDQVGAAKALLRLQDTYNLDTDTISKGNLPGVKHKSFLTAEDCFELGKVAYTEADYYHTELWMEQALRQLDEGEISTIDKVSVLDYLSYAVYQQGDLDKALLLTKKLLELDPEHQRANGNLKYFEYIMAKEKDVNKSASDDQSDQKTTPKKKGVAVDYLPERQKYEMLCRGEGIKMTPRRQKKLFCRYHDGNRNPKFILAPAKQEDEWDKPRIIRFHDIISDAEIEIVKDLAKPRLSRATVHDPETGKLTTAQYRVSKSAWLSGYENPVVSRINMRIQDLTGLDVSTAEELQVANYGVGGQYEPHFDFARKDEPDAFKELGTGNRIATWLFYMSDVSAGGATVFPEVGASVWPKKGTAVFWYNLFASGEGDYSTRHAACPVLVGNKWVSNKWLHERGQEFRRPCTLSELE

SEQ ID NO: 7 - DNA, 1956 bp, Homo sapiens
  CDS (30)..(1556)
  misc_feature (63)..(63): n is a, c, g, or t
ccgagcgccccgcctgctccgtgtccgacatgctgcgccgcgctctgctgtgcctgccgtggnccgccctggtgcgcgccgacgcccccgaggaggaggaccacgtcttggtgctgcggaaaagcaacttcgcggaggcgctggcggcccacaagtacccgccggtggagttccatgccccctggtgtggccactgcaaggctctggcccctgagtatgccaaagccgctgggaagctgaaggcagaaggttccgagatcaggttggccaaggtggacgccacggaggagtctgacctagcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggccgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccttggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacggcttccccacactcgggttctttcctgccagtgccgacaggacggtcattgattacaacggggaacgcacgctggatggttttaagaaattcctagagagcggtggccaagatggggcaggggatgttgacgacctcgaggacctcgaagaagcagaggagccagacatggaggaagacgatgaccagaaagctgtgaaagatgaactgtaatacgcaaagccggacccgggcgctgccgagacccctcgggggctgcacacccagcagcagcgcacgcctccgaagcctgcggcctcgcttgaaggagggcgtcgccggaaacccaaggaacctctctgaagtgacacctcacccctacacaccgtccgttcacccccgtctcttccttctgcttttcggtttttggaaaacccggatcctactctaggcagcccaccttggtgggcttgtttcctgaaaccatgatgtactttttcatacatgagtctgtccagagtgcttgctaccgtgttcggagtctcgctgcctccctcccgcgggaggttctcctctttttgaaaattccgtccgtgggatttttagacatttttacgacatcagggtatttgtt

SEQ ID NO: 8 - protein, 508 aa, Homo sapiens
  misc_feature (12)..(12): The ′Xaa′ at location 12 stands for Thr, Ala, Pro, or Ser.
MLRRALLCLPWXALVRADAPEEEDHVLVLRKSNFAEALAAHKYPPVEFHAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHGFPTLGFFPASADRTVIDYNGERTLDGFKKFLESGGQDGAGDVDDLEDLEEAEEPDMEEDDDQKAVKDEL

SEQ ID NO: 9 - DNA, 90 bp, Autographa californica nucleopolyhedrovirus
acaatatattatagttaaataagaattattatcaaatcatttgtatattaattaaaatactatactgtaaattacattttatttacaatc

SEQ ID NO: 10 - DNA, 71 bp, Autographa californica nucleopolyhedrovirus
ataaccatctcgcaaataaataagtattttactgttttcgtaacagttttgtaataaaaaaacctataaat